(19) United States
(12) Patent Application Publication
(10) Pub. No.: US 2020/0283348 A1
(43) Pub. Date: Sep. 10, 2020

(54) PROCESS FOR PRODUCING CYANOBACTERIA CONTAINING BIOFERTILIZER

(71) Applicant: Accelergy Corporation, Houston, TX (US)

(72) Inventors: Rocco A. Fiato, Basking Ridge, NJ (US); Attilio Bisio, Westfield, NJ (US)

(21) Appl. No.: 16/885,049

(22) Filed: May 27, 2020

Related U.S. Application Data
(62) Division of application No. 15/278,513, filed on Sep. 28, 2016.
Application No. 15/278,513 is a division of application No. 13/316,546, filed on Dec. 11, 2011, now abandoned.
(60) Provisional application No. 61/422,645, filed on Dec. 13, 2010.

(51) Int. Cl. C05F 11/08; C05F 11/02; C12M 1/00; C05F 17/20; C05F 17/40; C10G 1/00; C10G 1/06; C10J 3/72

(52) U.S. Cl. CPC C05F 11/08; C10J 2300/1656; C12M 21/02; C12M 43/06; C05F 17/20; C05F 17/40; C10G 1/002; C10G 1/06; C10J 3/72; Y02E 50/343; Y02W 30/47; Y02W 30/43; Y02P 20/134; Y02P 20/145; Y02E 50/32; Y02P 30/10; C05F 11/02

(57) ABSTRACT

A method for producing a biofertilizer in which a cyanobacteria and other photosynthetic microorganisms naturally occurring in the type of soil to which the biofertilizer is to be applied are reproduced in a closed photobioreactor using byproduct CO₂, and the reproduced microorganisms are incorporated in the biofertilizer.

### FIELD OF THE INVENTION

The present invention relates processes for producing a biofertilizer containing cyanobacteria, and preferably including other photosynthetic microorganisms.

### BACKGROUND OF THE INVENTION

A number of problems have hampered widespread use of coal and other solid fossil energy sources including that, in the case of coal, at least half of the carbon in the coal is converted to CO₂, and thereby wasted. The fact that, heretofore, a large amount of greenhouse gas (GHG), particularly in the form of CO₂, is emitted as a waste product in processes for the conversion of coal and other solid fossil energy sources to useful products has caused such processes to be disfavored by many from an environmental point of view.

It has been proposed to at least partially overcome the GHG problem by capturing and sequestering the carbon dioxide by re-injecting it into subterranean formations. Such an arrangement has the disadvantages of being expensive, of further reducing the process energy efficiency, of requiring the availability of appropriate subterranean formations somewhere in the vicinity of the conversion facility, of concerns about the subsequent escape into the atmosphere of the carbon dioxide, and of the waste of the energy potential of the carbon content of the carbon dioxide.

It has been proposed to use byproduct CO₂ emissions produced by coal conversion facilities to make algae and oxygen. Lipids in the algae can then be converted directly to liquid fuels, and the residual biomass, or if desired, the entire algae can be processed in indirect conversion processes such as Fischer Tropsch, to produce hydrogen and liquid fuels.

### SUMMARY OF THE INVENTION

In accordance with one aspect of the current invention, byproduct CO₂ is used to reproduce algal biomass, preferably in a closed photobioreactor (PBR), that includes soil-based, nitrogen fixing cyanobacteria, also called blue-green algae, and preferably other photosynthetic microorganisms, which are then incorporated in a biofertilizer that imparts beneficial properties to the soil to which the biofertilizer is applied. Importantly, the cyanobacteria, and preferably other photosynthetic microorganisms, are cultured from the set of microorganisms that are already present in the soil or type of soil to which the biofertilizer is to be applied.

After inoculation of soil with a cyanobacteria-based biofertilizer, the algal microorganisms repopulate the soil through natural reproduction, using sunlight, and nitrogen and CO₂ from the atmosphere, at much higher concentration than originally applied to the soil, thereby substantially reducing, or even eliminating, the CO₂ footprint of the process producing the CO₂ on a lifecycle basis, and substantially increasing the fertility of the soil for plant growth. The biofertilizer soil application rates can range from one gram per square meter to greater than 25 grams per square meter depending on soil type and soil moisture. This provides a highly leveraged effect on soil (terrestrial) carbon sequestration and greatly increases the fertility of the soil. Starting with one ton of CO₂, the application of the biofertilizer can result, on a lifecycle basis, in several tens of tons of additional CO₂ being removed from the atmosphere and sequestered in the treated soil and in vegetation, crops and/or trees grown in the soil.

In accordance with a still further aspect of the invention, during times such as cloudy days or at night when there is not enough available ambient sunlight to drive the photosynthesis for producing algal biomass and photosynthetic microorganisms, produced CO₂ is stored until sunlight is available, e.g., by liquefying the CO₂ or by storing it under pressure in bladders that can be part of or adjacent to the PBRs being used to produce the algal biomass and photosynthetic microorganisms. Alternatively, it is also possible to illuminate the contents of the PBR during non-sunlit hours in order to maintain the productivity of the algal biomass and photosynthetic microorganisms.

### BRIEF DESCRIPTION OF THE DRAWINGS

FIG. 1 is a simplified flowchart of an embodiment of the invention involving direct coal liquefaction and production of fertilizer from photosynthetic microorganisms.

### DETAILED DESCRIPTION OF EMBODIMENTS

In accordance with a first embodiment of the process of the invention, CO₂, e.g., produced by a coal to liquid hydrocarbons conversion process, is captured and used to produce algal biomass including cyanobacteria and, preferably, other photosynthetic microorganisms in a PBR. The PBR system can involve closed or open reactor systems; with closed systems being preferred to enable maximum production of specifically selected strain(s) of cyanobacteria and photosynthetic microorganisms and to minimize water loss and the contamination from external sources of the cyanobacteria and photosynthetic microorganisms. There are a number of commercially available algal biomass and photosynthetic microorganism production systems. One preferred system is the closed PBR system described in published US patent application numbers 2007/0048848 and 2007/0048859, which are incorporated herein by reference in their entirety.

The cyanobacteria and photosynthetic microorganisms reproduced in the PBR can be isolated in aqueous streams for use as a soil treatment material in order to increase the carbon content of the soil and for inducing photosynthesis to self-replicate in the soil. The resulting microorganisms can also be dried and combined with other additives such as organic binders, alkali containing residues from the gasification and/or DCL facility and the final mixture used as a natural bio-fertilizer. In this capacity, the material not only results in further growth of such microorganisms in the soil via photosynthesis, thereby increasing its natural carbon content, but also causes various components of the cyanobacteria and other microorganisms to fix nitrogen, all of which promotes the growth of plant life in the treated soil and greatly reduces the GHG, and particularly the CO₂, footprint of the process of producing the CO₂. PBR systems suitable for the purposes of this invention include those described in provisional U.S. patent application, Ser. No. 61/422,613, the contents of which are incorporated herein by reference in their entirety, and those developed by BioProcess Algae, LLC., Phyco Biosciences, or Solix BioSystems.

In accordance with the invention, the naturally occurring complement of microorganisms, including cyanobacteria, occurring in the soil or type of soil to which the biofertilizer is to be applied is optimized and amplified in a closed PBR and the resulting material is dewatered and dried and treated with desirable additives; after which it is granulated, optionally coated with materials to optimize its spreading characteristics and distributed on the soil that is to be fertilized or restored. Alternatively, microorganisms that include one or more strains of cyanobacteria and other components compatible with the type of soil and environmental conditions where the biofertilizer is to be applied, are amplified in a closed PBR to generate the material for the biofertilizer.

The process of the invention has the extremely important advantageous characteristic that because the set of cyanobactraia and other microorganisms applied to the soil is specifically selected to be compatible with the makeup of the soil to which it is applied, it multiplies, e.g., through photosynthesis, thereby extracting more CO₂ from the atmosphere and fixing atmospheric nitrogen. This characteristic results in an increase in the net CO2 sequestered by a factor of 30 and potentially as much 150 fold over the CO2 consumed during the production of the microorganisms in the ICBTL process of the invention, and greatly enriches the fertility of soil. The biofertilizer can also be mixed with the soil as a soil amendment.

The quality of the natural bio-fertilizer, as affected by the quality of the water and the purity of the CO₂ and other nutrient streams provided to the PBR from the process producing the CO₂, can be controlled to generate food grade/FDA certified material for use in enhancing growth of various food crops; to an intermediate grade to serve as a soil amendment material for reclamation of arid soils to prevent or inhibit wind erosion via formation of a bio-active crust; or to lower purity material for use in reclamation of spent mine soils where the addition of a bio-reactive material inhibits leaching and erosion of contaminated soils to improve the quality of water drain off.

The natural bio-fertilizer can also be used as a direct replacement for conventional ammonia based fertilizer, where it offsets large amounts of CO₂ that would otherwise be generated in production of NH₃ and the full range of ammonia based fertilizers. This also leads to other downstream benefits, such as a reduction in run off of NH₃ based components that contaminate downstream waterways and cause unwanted blooms of algae and other aquatic plants.

In order to minimize the CO₂ footprint in the system of the invention and convert substantially all of the CO₂ to cyanobacteria and other photosynthetic microorganisms, the produced CO₂ can be stored during periods of low light or darkness when there is not enough light for photosynthesis to produce cyanobacteria and other photosynthetic microorganisms from the CO₂. Alternatively, microorganism production can be continued using artificial light sources. Coupling these steps together allows for recovery and reuse of the equivalent of as much as 270 times the CO₂ conversion to algae alone using an open pond or PBR without the use of artificial light. Without storage, the quantity of CO₂ reused is reduced by a factor of three or four. Techniques for storage of CO₂ include liquefaction of the CO₂, conversion of the CO₂ to ammonium bisulfide or urea by well-known conventional chemical processes, physical storage and others.

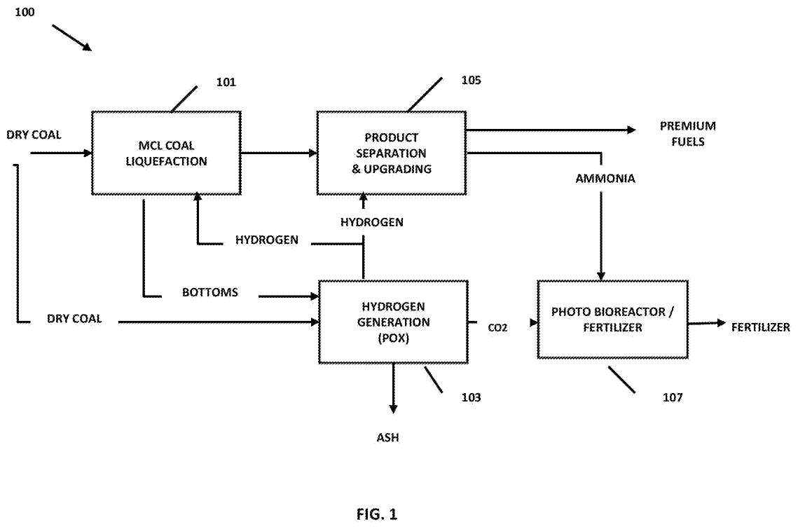

Referring now to FIG. 1 of the drawings, there is illustrated a coal conversion system 100 that produces a maximal amount of biofertilizer so as to have an extremely small and even negative carbon footprint. Coal is fed to the Direct Coal Liquefaction (DCL) reactor system 101 and to the partial oxidation (PDX) system 103. The coal fed to the DCL reactor system 101 is liquefied and the products thereof are fed to the product separation and upgrading system 105 to generate premium fuels such as gasoline, diesel and jet fuel and/or chemical feedstocks. Bottoms from the DCL reactor system 101 are fed to the PDX system 103, in which they are gasified to generate hydrogen for supply to the DCL reactor system 101 and the product separation and upgrading system 105. The PDX system 103 also generates large amounts of concentrated, pure CO₂ which is supplied to the photobioreactor algae and formulated biofertilizer production system 107 that preferably includes one or more closed PBR's such as described in the references identified above. Ammonia from the product separating and upgrading system 105 is also supplied to the photobioreactor algae and formulated biofertilizer production system 107 as a nutrient. The system 107 preferably produces cyanobacteria and other photosynthetic microorganisms for use in a biofertilizer having the CO₂ terrestrial sequestration and nitrogen fixation advantages described above. In the PDX process, coal is partially combusted, non-catalytically, in a gasifier with oxygen typically at 2,600° F. and 1,000 prig. At these conditions, the ash is converted to a liquid and flows down the inside wall of the gasifier and is collected at the bottom of the gasifier. The syngas that leaves the top of the gasifier is scrubbed of particulate matter and carbon monoxide present in the syngas can be converted to hydrogen via the water-gas shift reaction. Sulfur and CO2 are removed in a double-stage Selexol Unit.

A number of commercial vendors including Shell, Siemens, and General Electric have applied PDX commercially and offer the technology for license. UOP and others license the Selexol Process.

In the production of the biofertilizer, a PBR is inoculated with a biological culture drawn from its normal residence in the top centimeter of healthy undisturbed soil found in un-shaded areas having similar soil and environmental characteristics as the soil to which the biofertilizer is to be applied, or with a biological culture that includes one or more cyanobacteria strains and preferably other photosynthetic microorganisms suitable for use as a fertilizer in the location where the biofertilizer is to be used. Such cyanobacteria can be referred to as “soil-based cyanobacteria.” In nature, these soil microorganisms form a biological soil crust (“BSC”) that serves many functions, including gluing the soil grains in place, thereby limiting wind and water erosion, as well as providing fertilization and plant vitality.

Cyanobacteria and “cyanolichens” are a primary source of fixed atmospheric nitrogen in arid ecosystems. Studies, in the western United States, have observed that between 5 to 49 cyanobacterial taxa depending on the study site. Nostoc, Schizothrix, Anabaena, and Tolypothrix are the most frequently encountered heterocystous genera. Microcoleus and Phormidium are commonly encountered non-heterocystous genera. In western Colorado, for example, Scytonema, a heterocystous genus, is frequently observed. Heterocysts are differentiated specialized cells responsible for nitrogen fixation. Heterocysts lack the water-splitting O.sub.2-evolving Photosystem II apparatus. This adaptation has evolved to eliminate the inhibition of nitrogenase activity by O.sub.2, but still generates ATP energy by retaining photosystem-I activity.

Many non-heterocystous cyanobacterial genera are known to contain nitrogenase and may fix nitrogen in the dark under microaerophillic or anaerobic conditions. Microcoleus vaginatus is an extremely important microbiotic crust component based on its frequency of occurrence and morphology. The mucilaginous encased filaments of Microcoleus vaginatus are highly effective in binding sand particles, thus reducing erosion and producing a stable substrate for the colonization of cyanolichens and other microorganisms. Although Microcoleus vaginatus may not fix nitrogen directly, it is thought that its mucilaginous sheath provides an anaerobic micro-environment and carbon source for epiphytic diazotrophic bacteria.

Cyanolichens are also a major contributor of fixed-nitrogen and microbiotic crust ground cover in desert ecosystems. Lichens are a mutualistic symbiosis between a fungus (mycobiont) and an alga (phycobiont). In most cases, the lichen phycobiont is a green alga, usually Trebouxia, but the cyanolichen phycobiont consists of cyanobacteria, most commonly Nostoc, Scytonema, or Anabaena. These cyanolichens are characteristically black, gelatinous in texture, and non-stratified. Certain stratified lichens inhabiting subalpine biomes, such as Peltigera and Lobaria, contain both the green Trebouxia, and the nitrogen-fixing cyanobacterium, Nostoc. For example, the cyanolichens of the arid western United States can occupy from 40 to 100% of the ground cover and make significant contributions towards soil stabilization and N.sub.2-fixation. Depending on the soil and abiotic environment, up to 159 lichen species representing 53 genera have been observed. Some of the most commonly encountered genera include, Collema, Placinthium, Leptogium, and Heppia.

The cyanobacterial genera to be exploited may be obtained from biological soil crusts and include, but are not limited to the following genera: Nostoc, Anabaena, Scytonema, Tolypothrix, Calothrix, Microcoleus, Rivularia, Phormidium, Symploca, Schizothrix, Stigonema, Plectonema, and Chroococcus. In addition to these cyanobacteria, it can be desirable to include eukaryotic algae such as Chlamydomonas, Trebouxia, Scenedesmus, for instance. In many cases, it will be desirable to include free-living nitrogen-fixing bacteria, such as Azotobacter, Rhodospirillium, or Rhodopseudomonas, for example. Other important soil bacteria such Arthrobacter and various actinomycetes including the genera, Frankia, Nocardia, Streptomyces, and Micromonospora may be included to enhance nutrient cycling. Finally, it may also be desirable to include lichenizing, saprophytic, and mycorrhizal fungi to complete the microbial complement of the basic photosynthetic biofertilizer. These heterotrophic microorganisms will be produced using standard methods.

The biofertilizer is preferably designed, in addition to providing soil nitrogen and carbon, to behave as an erosion control agent. In most cases, the biofertilizer alone will achieve the desired results. Based on the flexibility of the biofertilizer, it can be used in conjunction with traditional erosion control methods such as fibrous mulches and tackifiers thus enhancing the efficacy of these traditional products. For instance, hard-rock mine tailings, waste and overburden characteristically become acidic (pH<3) through the oxidation of sulfur by bacteria. These acidic environments inhibit seed germination, and exceeds the lower pH limit of cyanobacteria (pH<5). However, it has been shown that when a layer of mulch is applied to the surface, it serves as a chemical insulator that permits seed germination and the growth of the biofertilizer. The plant roots penetrate into the nitrogen-deficient acidic mine tailings and continue to grow when nitrogen is supplied by the biofertilizer.

It has it has been found that rhizobacteria are a key component of the microorganisms found in soils. It is believed that cyanobacteria particularly when present in combination with rhizobacteria act as a phyto stimulator and generate organic acids including gibberellic acid and acetic acid and other mono and poly carboxylic acids, which are important stimulants for plant growth. It has further been found that different kinds of soil formation have different complements of naturally occurring microorganisms that contribute to to the fertility of the soil for various crop and natural plant species to take root and flourish. For example, the Desert Institute of the Chinese Academy of Sciences has found in desert soils that, in sand, the primary surface layer microorganisms were found to be Fragilaria, Oscillatoria willei, and Phormidium okenii. Where the surface layer is an algal crust the primary microorganisms were found to be Synechococcus parvus, Tychonema granulatum and Phormidium retzli. Where the surface layer is a lichen crust the primary microorganisms were found to be Oscillatoria wille, Oscillatoria carboniciphila and Phormidium retzli. In the case of the moss crust surface layer, the primary microorganisms were found to be Synechococcus parvus, Synechocystis pavalekii and Phormidium retzli. It is particularly beneficial to nurture such natural colonies to form, particularly in arid regions were reestablishment of natural flora can be beneficial to soil stabilization and to the increased production of natural plant colonies in replenishing the soil with carbon and other nutrients. The Institute has reported that certain species of these microorganisms are prevalent in soil samples in the Gobi and nearby deserts in China, and these species are of particular interest as potential members of the population of organisms to be incorporated into the final biofertilizer formulation of this invention. For example, see the recent report by Yanmei Liu et al on “The Effects of Soil Crusts on Soil Nematode Communities Following Dune Stabilization in the Tennger Desert, Northern China” Applied Soil Ecology, vol 49, pp 118-124 (2011).

Many of the microorganisms in the BSC are also photosynthetic and draw their energy from sunlight such that they can, in-turn, manufacture and provide nutrition and fixed nitrogen to cohort microorganisms that are not photosynthetic or are found deeper in the soil. The actions of the BSC, and the deeper cohort microorganisms it supplies nutrition to, work together to stabilize soil and draw plant available nutrition from the grains of soil into the soil matrix over time. In addition, the dominant cyanobacteria component of BSC fixes carbon as well as nitrogen from the atmosphere. Beginning with BSC, the combined actions of these microorganisms create conditions benefiting the establishment and growth of vascular plants like grasses, shrubs and crops. In effect, the BSC is a naturally occurring solar powered fertilizer that lives on the surface of bare earth making it suitable and beneficial for the establishment of vascular plants over time. However, because BSC microorganisms reproduce slowly in dry climates and are not very motile, physical disturbances like tilling, livestock grazing, and fire can halt the BSC's beneficial effects for the soil and the BSC, and these benefits can take decades or centuries in dry climates to naturally restore. The production of the preferred biofertilizer rapidly reproduces naturally occurring BSC microorganisms at an industrial scale in a PBR. The microorganisms are then carefully compounded to form “inoculant seeds” of these microorganisms that constitute the preferred biofertilizer, and that are spread onto land presently lacking healthy soil crust colonies, thus accelerating the natural recovery of the soil. As the biofertilizer propagates on the soil surface, it draws down increasing amounts of carbon from atmospheric CO₂ into the soil where that carbon becomes part of a living sustainable microbiological community and effectively sequesters this atmospheric carbon into the soil. Through soil inoculation with the preferred biofertilizer, its natural propagation on the soil and secondary vascular plant growth enhancement, it has been estimated that the conversion of 1 ton of CO₂ into the preferred biofertilizer, which is then applied onto suitable soils, can cause the drawdown of up to 50 tons of CO₂ from the atmosphere annually through direct photosynthetic uptake of atmospheric gasses by that soil.
The cyanobacteria and their soil consortia used to produce the biofertilizer are preferably cultured into an inoculum in a manner taught by U.S. Patent Application Publication No. US 2008/0236227 to Flynn, the contents of which are hereby incorporated by reference in their entirety, (herein after referred to as “Flynn”) and used to inoculate an amplifying PBR, also taught by Flynn, where the culture can be rapidly grown in liquid media via ready access to nutrients, carbon dioxide, sunlight and hydraulic mixing. The PBR may be fed by sunlight, nutrients and a carbon source that is most commonly carbon dioxide, but that may be a fixed form such as sodium bicarbonate or other bio-available forms.

A preferred method for producing the biofertilizer in accordance with the present invention includes the following steps:

(1) Isolating the important photosynthetic biological soil crust microorganisms to produce a polyspecies culture that closely reflects the native microbial species composition;

(2) Cultivating the culture in a PBR, preferably under controlled conditions designed to maximize biomass productivity;

(3) Harvesting the produced biomass by, for example, a simple gravity-driven sedimentation and filtration, clarification, or centrifugation;

(4) Preserving the biomass by, e.g., using refractance window drying technology, or other methods such as air drying, spray drying, vacuum drying, or freezing such that the cells remain viable;

(5) Pulverize, flake, or powder the dried cyanobacteria to facilitate packaging, storage, shipment, and final dissemination of the biofertilizer. After growing in the PBR, the soil microorganisms being harvested and compounded using admixes and coatings to create the product biofertilizer, the biofertilizer can be spread upon farmlands or damaged land using standard agricultural practices, such as crop dusting, mixing with irrigation water or applying with spreading machines. Once on the soil surface, the natural availability of carbon dioxide and nitrogen in air, along with available participation or irrigation water and sunlight, causes the biofertilizer to induct a growing colony of soil microorganisms in proportion with the suitability of growth conditions for that specific consortium of microorganisms. The consortium of microbes in a locally adapted biofertilizer is preferably picked from local soil samples representing the best target outcome that could be expected from a soil crust reseeding effort of similar local soils. When this is done and the biofertilizer is spread to sufficient surface density, then the crust will reestablish at an accelerated rate well in advance of natural propagation. In land reclamation efforts, sufficient application density is approximately 0.1 to 2 biofertilizer particles per square cm. In agricultural applications where accelerated fertilization performance is required, sufficient application density is approximately 1 to 20 biofertilizer particles per square cm.

As microorganisms grow and propagate in and on the soil, their uptake of CO₂ from the atmosphere increases proportionate with the population size, impinging sunlight, water availability, soil type and the occurrence of secondary vascular plant growth that might further increase the net primary productivity of the soil. The amount of CO₂ drawn down from the atmosphere will vary widely dependant on these factors. It is estimated that if a crust is allowed to grow to maturity in a land reclamation application, that it will draw down from the atmosphere approximately 100 grams of CO₂ per square meter per year.

The soil sample is preferably drawn from a desired target outcome soil patch that represents the best and most desired microbiological outcome for the treated soil, and that is similar in non-biological constitution and environmental factors to the soil in the area to be treated. In this way, a consortium of microorganisms can be specifically selected to manufacture a particular regional type of biofertilizer that includes microorganisms most favored to survive, thrive and fertilize on the targeted soil to be treated in that region.

The purpose of the inoculation PBR is to obtain the organisms from the target outcome soil and begin growing a population facsimile within the PBR's liquid medium. The population generated by the inoculation PBR should have substantially the same or otherwise sufficient microorganism consortia members and in roughly substantially the same or otherwise sufficient balance as they were present natively in the soil. The PBR operator uses input and output population and growth media assay data to adjust growth input parameters such as light, pH, temperature, CO2 and nutrient levels, as well as mixing speed to effect the desired growth rate and population balance characteristics on the output of the incubator. In a similar fashion, the amplifier and production PBR operator looks at the population and growth media assay between the input and output of the PBRs and adjusts the same growth conditions to effect the desired result. In some cases, the desired product population ratio may be different from that found in the target outcome soil, but will affect a better result upon application via that difference.

The pH and rate of photosynthesis in the PBR system can be measured using the PT4 Monitor, available from Point Four Systems Inc. (Richmond, British Columbia Canada), which includes the controller, acquisition software, dissolved oxygen, pH, and temperature probes. The difference in dissolved oxygen between the lower and upper probe arrays provides a measure of photosynthesis. Likewise, the difference in pH between the lower and upper probe arrays is a measure of CO₂ consumption. Under illumination, the microorganisms will photosynthesize and assimilate CO₂ causing the pH of the medium to rise. When the pH increases to a chosen set point, preferably pH 7.5, the controller will introduce 100% CO₂ into the PBR, which will cause the pH to drop as a result of the formation of carbonic acid and related complexes.

The output of the PBR may be fed into filtering and drying belts in which various optional admixes can be applied. The resultant dry flake and its optional coating may then be granulated to become the biofertilizer. The final biofertilizer product can be distributed and applied to soil via various agricultural and land restoration spreaders. Advantageously, the biofertilizer pellets can be broadcast by a spinning spreader or aircraft such that they are not blown away by the ambient wind. The biofertilizer can also be mixed with irrigation water and sprayed on crops. The various admixes optionally to be included also desirably remain physically associated with the microorganism consortium in the same relative proportions, even as the composite admix/biomass flake is reduced in size by granulation. By even layering and infusing of the admix homogeneously across the flake as the flake is being generated, then these relative proportions of admix/biomass can be maintained during the granulation and particle coating process. The dry admix components may be further added as the biomass mat begins to consolidate, which helps to mechanically consolidate them with the biomass by entrapping some of the dry admix in the filaments of the consolidating cyanobacteria. The wet admix is typically, but not exclusively, a sugar based composition of xero-protectants and heterotrophic consortium member nutrition additive that serve to bind and glue all the components together as it dries. Using an actual mucilage or other water soluble glue for this purpose, or a solvent based but UV degradable binder, can also be considered for this purpose.

The following are optional admixes and their purpose:
1) Anti-oxidants such as beta carotene can preserve the biofertilizer during the drying process and in storage.
2) Xero-protectants such as sucrose and other sugars, or a biologically derived xero-protectant called trehalose can prevent cell damage from rapid desiccation and extended desiccation over time.
3) Growth nutrients include micro nutrients needed by all soil microorganisms as taught by Flynn including sugars to feed the non-photosynthetic cohorts during the initial stages of establishment.
4) Sand or clay fillers serve two purposes. One is to increase the weight density of the resultant granulated particles thereby making them more aerodynamically spreadable from aircraft and land based spreaders and resistant to wind currents. The other purpose is to provide a non-damaging location for fracture lines between the desiccated microorganisms during granulation that does not split through the microorganism itself.
5) Spread pattern tracers may be fluorescent additives. Another tracing tag may be the use of inheritable but non-operational unique gene sequences within one of the microorganisms that will propagate at the same rate and with the same spatial characteristics as the biofertilizer propagates. This will allow a researcher or carbon credit auditor to visit a patch of soil months or years after initial application of the biofertilizer and know how much of the soil crust or under-earth biomass is directly due to the propagation and beneficial actions of the specifically tagged biofertilizer.
6) Vascular plant seeds like restorative grasses or actual crop seeds may become part of admix. In this case the biofertilizer would be designed to work in biological concert with the embedded vascular plant seeds to achieve and maximize the desired restorative of fertilizing result.
7) A tackifier may be added to the admix in order to quickly bind the particle with other soil grains upon first environmental wetting to prevent further shifting by wind or water erosion.
8) Other microorganisms may be added to either the dry mix or to the wet mix. These other microorganisms may be chosen for their auxiliary properties like being a good tackifier or they may be chosen because they are an important part of the biological consortium of the biofertilizer; yet for various reasons such as growth media type incompatibility or susceptibility to predation they were not able to be co-grown in the same PBR as the rest of the biofertilizer consortium members.

Biologics may also be spray coated onto the exterior of the particle. In this context “biologics” can refer to whole living or dead cells or bio-active substances that affect the receptivity of the soil to being colonized by the biofertilizer microorganisms. Alternatively, these substances may be intended to prevent the consumption or destruction of the biofertilizer by other living organisms such as insects, other microorganisms, birds or other living creatures.

What is claimed is:

1. A method for producing a biofertilizer comprising the steps of:
a. inoculating a photobioreactor (PBR) with inoculant including a nitrogen fixing cyanobacteria naturally occurring in the type of soil to which the biofertilizer is to be applied;
b. supplying CO₂ to said PBR for reproducing the inoculated cyanobacteria therein; and
c. ncorporating the reproduced cyanobacteria in the biofertilizer.

2. The method of claim 1 wherein the inoculant further includes one or more additional microorganisms selected from the groups comprising free-living nitrogen-fixing heterotropic bacteria, actinomycetes, photosynthetic bacteria, mycorrhizal or lichenizing fungi, and combinations thereof.

3. The method of claim 1 wherein said inoculant is obtained from the soil to which said biofertilizer is to be applied.

4. The method of claim 2 wherein the nitrogen-fixing heterotropic bacteria are selected from the Azobacteriaceae or Frankiaceae groups comprising Azotobacter, Frankia, or Arthrobacter.

5. The method of claim 2, wherein the photosynthetic bacteria are selected from the Rhodospirillales group comprising Rhodospirillium, Rhodopseudomonas, and Rhodobacter.

6. The method of claim 2, wherein the mycorrhizal fungi belong to the Glomales, and the lichenizing fungi belong to the groups including one or more of Collema, Peltigera, Psora, Heppia, and Fulgensia.

7. The method of claim 1, further including transforming the biofertilizer into a dormant state by a technique selected from the group consisting of spray drying, refractance-window drying, solar drying, air drying, or freeze drying.

8. The method of claim 7 where xeroprotectant additives including one or more of sorbitol, mannitol, sucrose, sorbitan monostereate, dimethyl sulfoxide, methanol, .beta.-carotene, and .beta.-mercaptoethanol are used to increase post drying viability.

9. The method of claim 1, wherein the biofertilizer is applied in combination with an additive selected from the group consisting of fibrous, cellulosic mulch material, polymeric tackifiers, clays, geotextiles, and combinations thereof.

10. The method of claim 1 wherein said inoculant further includes rhizobacteria.

11. A method for producing a biofertilizer comprising the steps of:
a. reproducing in a PBR with the use of byproduct CO₂, nitrogen fixing cyanobacteria naturally occurring in the type of soil to which the biofertilizer is to be applied; and
b. incorporating said cyanobacteria in the biofertilizer.

12. The method of claim 11 wherein said cyanobacteria is obtained from the top centimeter of the soil to which said biofertilizer is to be applied.

13. The method of claim 11 wherein said PBR is a closed PBR.

14. The method of claim 11 wherein other photosynthetic microorganisms are reproduced in said PBR together with said cyanobacteria.

15. The method of claim 11 wherein other photosynthetic microorganisms include rhizobacteria.
